(19) United States
(12) Patent Application Publication
(10) Pub. No.: US 2024/0382446 A1
(43) Pub. Date: Nov. 21, 2024

(54) METHOD FOR IMPROVING FEED INTAKE

(71) Applicant: OREXA B.V., Herpen (NL)

(72) Inventors: Bernardus Wijnand Mathijs Marie PEETERS, Herpen (NL); Antje Johanna ZONNEVELD, Herpen (NL)

(73) Assignee: OREXA B.V., Herpen (NL)

(21) Appl. No.: 18/686,681

(22) PCT Filed: Sep. 1, 2022
(86) PCT No.: PCT/EP2022/074366

§ 371 Date: Feb. 26, 2024

(30) Foreign Application Priority Data
Sep. 1, 2021 (NL) 2029094

(51) Int. Cl. A61K 31/245; A23K 20/111; A23K 50/30; A23K 50/60; A61K 9/00; A61P 3/02

(52) U.S. Cl. CPC A61K 31/245; A23K 20/111; A23K 50/30; A23K 50/60; A61K 9/0056; A61P 3/02

(57) ABSTRACT

The invention provides a method for improving the feed intake of domesticated animals such as husbandry animals and pets, and humans, more particularly for pigs and piglets. The method is directed to offering of a composition that comprises benzocaine and feed ingredients that have been adjusted to the needs of the animal concerned. It also provides a composition suitable for use in the method according to the invention.

This invention relates to a method for increasing feed or food intake and a composition specifically suitable therefore, more particularly a method for increasing feed intake of domesticated animals such as husbandry animals, pets, and humans and a composition suitable therefore. The method comprises mixing benzocaine in the feed.

### BACKGROUND

This invention addresses the purpose to improve feed or food intake, particularly the feed or food intake of domesticated animals such as husbandry animals, pets, and humans, more particularly in pigs, and other infant husbandry animals. It is of economic interest in husbandry that the animals, in particular swine, grow readily. In the present disclosure pigs, pig weaning, pig feed and piglet weaning feed is described as an example, but the particulars mentioned here may also suitably be applied to other husbandry animals (such as cattle, goats, sheep, and horses), pets and humans. Pigs need a high energy diet that is low in fibers and contain ample protein. Pigs will consume enormous amounts of feed quickly. To raise and maintain a healthy stock, maximize growth and reproduction, and increase production, it is necessary to feed them the right food and a balanced diet from wean to finish. Because a pig eats approximately 4% of its body weight per day, they require a number of essential nutrients to meet their daily needs, i.e., water, carbohydrates, fats, protein (amino acids), minerals, and vitamins.

With a proper diet weight gains of about 0,68 kg to 0,77 kg per day up to a weight of approximately 50 kg can be expected. After that, weight gains of 0,82 kg to 1,0 kg per day are achievable. Ingredients for pig feed can be among others: wheat, wheat middlings, barley, barley flower, rye, rye middlings, maize, maize flour, potato protein, beet pulp, molasses, forage meal of alfalfa, fodder beet, sunflower seed scales, rapeseed scales, linseed scales, palmkernel scales, cotton seed, tapioca, citrus, maize gluten, maize flower, maize fodder flour, oats, peas, broad and field beans, soya scales, rapeseed scales, linseed, corn cob mix (CCM), triticale, soya beans and soya scales.

Swine food for husbandry should not contain high-sugar foods, dog food, cracked corn, milk, fish, meat, fruits or potatoes, the latter unless processed. Foods high in sugars can slow growth rates while milk, meats, and fish can harbor viruses. Pits and seeds of apples, pears, apricots and peaches contain a cyanogenic glycoside that is released when chewed causing illness. Potatoes contain natural toxins which can cause severe stomach ache.

It is of paramount importance to implicate that proper food supply depends on the growth stage of the pigs and other conditions, such as pregnancy.

Piglets, younger pigs that weigh less than 18 kg, should be introduced to a solid diet through weaning feed (also called creep feeding) while they are still suckling. Dietary needs will increase daily with a pig's weight.

Growing pigs, those weighing 18 kg to 60 kg, and finishing pigs, those weighing 60 kg to market weight (approximately 100 kg or more) should transition from the grower feeds that are nutrient dense with more protein to finisher feeds that are less dense.

In the weeks after birth piglets grow rapidly, but within three weeks, a piglet's nutritional requirement outpace the ability of the sow to provide it. In fact, a sow's milk production peaks in less than a month and then slowly declines. The most important aspect in this phase is to prepare the digestive system for weaning and the intake of feed, which contains mainly ingredients originating from plants.

Therefore, from pre-weaning in the farrowing room to post-weaning in the nursery, creep feeding is used to increase piglets' nutrient intake and familiarize them with solid food prior to weaning. Creep feeding is a transition strategy in swine production designed to successfully introduce piglets to a solid diet and prepare their digestive system for weaning. Offering creep feed in the first week of life next to the sow enables the piglets learning to eat solid feed. Feed intake is very low in the first two weeks of life, as milk makes up the majority of the diet.

Physiologically, piglets are not yet ready to digest solid feed. Here, small amounts of feed ingested make a difference: piglets learn there is more available than milk and the digestive tract is stimulated to digest the unknown materials by triggering the production of the necessary enzymes.

Though creep feeding cannot guarantee greater weaning weights, the main benefit comes from enhanced post-weaning feed intake and better development of the gastro-intestinal tract. Because weaning is such a stressful period for the piglet, creep feeding can prepare the gastro-intestinal tract and improve the overall health and well-being as it enters the nursery. Recent research indicates that with higher quality creep feed and subsequent weaner nutrition, pigs perform and feel better. Creep feeding prepares the gastro-intestinal tract of piglets for intake of solid food.

In nature, sows wean piglets gradually over 12 weeks. In current production systems, piglets are weaned between day 21 and 28 of life. At this age, piglets are rather vulnerable because neither the immune system, nor the digestive system is fully developed.

It is known that the higher the feed intake during the first week of post-weaning, the better the overall growing-finishing performance. A difference of 50 to 100 grams per day in the first week post-weaning can gain even a whole week from reaching market age. However, even though piglets can consume up to 300 grams feed per day during the first week post-weaning, their genetic potential is not exhausted here. Today, a good quality diet used properly can support up to 200 to 250 grams daily feed intake. The target can be to reach and even exceed 300 grams.

From week 3 onwards, the intake of starter feed increases steadily. Due to the enormous growth potential in this phase and declining milk production of the sow, supplemental feeding can provide the necessary nutrients to increase piglet growth.

Weaning is a stressful process for piglets, because they are removed from their mother, maybe even their littermates. They are moved to a new stable with new feeding technique and climate, and are mixed with piglets from other litters. Additional stress comes from losing access to their main feed source, sows' milk, from one day to the next, which forces them to ingest solid feed with a very different composition compared to sows' milk. The older the piglet, the more stable it is and the better it copes with these challenges.

After weaning maintaining feed intake is the most important factor to avoid the commonly known “weaning dip”.

A schematic view of such a weaning dip is depicted in FIG. 5.

A stable feed intake after weaning avoids that starving piglets start to overeat after some days and overload their gut with feed that the piglet cannot digest efficiently. Pathogens in the gut will thrive on the undigested nutrients, which often results in scours and edema disease.

Moreover, several studies demonstrated that piglets having a good growth performance right after weaning, also show better performances until slaughtering.

The best measure to keep the piglets eating after weaning is preparing them ahead of time. In that case piglets know how to eat and the gut is able to digest the solid feed more effectively. Nevertheless, the gut is still developing and needs support to overcome the stressful weaning phase. Feed for weaned piglets must fulfil the same high quality standards as weaning feed.

In U.S. Pat. No. 7,754,234 a composition is disclosed comprising an antacid, and a local, topical anesthetic. The composition is used to relieve pain or discomfort associated with a sore throat, and therefore, the invention is also directed to a method of alleviating the pain or discomfort associated with a sore throat comprising instructing a human to orally administer the composition. This disclosure describes the use of lidocaine, benzocaine, tetracaine, or dyclonine in combination with starch paste and optionally sweeteners.

The composition is solely concerned with relieving pain of discomfort, not with providing nutritious food at the same time, nor with increasing feed intake.

U.S. Pat. No. 10,828,272 discloses a method to facilitate food intake and food retention in a mammal by inserting a fluid local anesthetic into the lumen of the esophasus of the patient via a gastrointestinal tube. The local anesthetic can be amethocaine, articaine, benzocaine, bupivacaine, chloroprocaine, cinchocaine, cyclomethycaine, dibucaine, diethocaine, etidocaine, larocaine, levobupivacaine, lidocaine, lignocaine, mepivacaine, novocaine, piperocaine, prilocaine, procaine, proparacaine, propoxycaine, QX-222, QX-314, ropivacaine, tetracaine, trimecaine, menthol, eugenol or a safe pharmaceutical formulations of tetrodotoxin, saxitoxin, or neosaxitoxin.

The disclosure is useful for patients having problems with food intake or food retention. This disclosure is concerned with a medical treatment, performed by a medical specialist rather than with a method that can be used by farmers, breeders and pet owners.

US 2020/0376026 discloses a method to improve efficiency, growth, and performance in an animal by orally administering to the animal an effective amount of a therapeutic clay.

The terms used in this specification are used according to their usual meaning. In case of doubt our use of the terms can be further understood as follows:

In the description the term “feed” is used for “feed and food” collectively or feed only. If solely human consumption is meant the term “food” is used. Both terms are directed to those ingredients that are commonly used in daily food intake of the animal or human concerned and have nutritional value.

In the present description the term “creep feed” and “weaning feed” are used interchangeably. It refers to the feed that is especially adapted to help an infant animal adjust to solid food rather than mother milk. This type of feed is commercially available, but can also be composed by hand.

Facilitation of food or feed intake and food or feed retention does not only mean an increase in the volume of an eating bout, but also of an increase in the total volume of feed or food intake during a within-one-day interval by shortening of interval(s) between eating bouts or shortening of time spent on an eating bout with concomitant increase in frequency of feeding bouts.

### SUMMARY

The present invention contributes to improved results in husbandry animal breeding by providing a method to improve feed intake in a domesticated animal by offering feed in the form of the composition wherein the composition comprises benzocaine and feed ingredients which have been adjusted to the specific needs of the animal concerned.

The method is specifically suitable for the weaning of piglets, since especially piglets and pigs may benefit from a smooth and problemless weaning period.

However, it may also suitably be used for other domesticated animals such as pets and for humans, provided that the feed ingredients have been adjusted for consumption of the animal or human concerned.

The present description is also directed to a method to improve feed intake of husbandry animals such as a piglet or a pig, a calf or cow or bull, a sheep or a lamb, a horse or a filly by offering feed in the form of a composition comprising benzocaine and feed ingredients according to the description

Preferably the feed ingredients have been adjusted to the specific needs of infant animals such as piglets or for pigs. Thus for infant animals such as piglets the feed preferably is weaning feed and is offered to piglets during the weaning period.

Preferably the benzocaine is present in the feed in a solid form, more preferably in solid particulate form. The solid particulate form facilitates the dosing and storage of the benzocaine.

Usually the feed comprises milk products, fibers and high quality protein source.

In addition thereto the feed may comprise feed additives such as enzymes, acidifiers or organic acids, probiotics, phytogenic feed additives or botanicals, and/or toxin binders and combinations thereof.

The amount of benzocaine present in the composition is preferably calculated to provide a dosage of 0.2-20 mg benzocaine per day per kg animal.

When the feed is provided to a pig or piglet the amount of benzocaine present in the feed composition is preferably calculated to provide a dosage of 10-1000 mg benzocaine per day per piglet or pig.

The present disclosure is also directed to a composition specifically suitable for use in the method according to the invention. Said composition comprises benzocaine and feed ingredients for domesticated animals or humans and benzocaine which have been adjusted to the specific needs of the animal concerned.

The benzocaine in the composition preferably is present in a solid, particulate form so as to facilitate dosing and storage.

The composition according to the present disclosure preferably contains feed that comprises milk products, fibers and high quality protein source.

### DETAILED DESCRIPTION

As described above, the present description is directed a method to improve feed intake in a domesticated animal by offering feed in the form of the composition wherein the composition comprises benzocaine and feed ingredients which have been adjusted to the specific needs of the animal concerned.

The method may also suitably be used for other domesticated animals such as pets and even for humans, provided that the ingredients have been adjusted for consumption of the animal or human concerned.

With domesticated animals both husbandry animals and pets are meant. With husbandry animals such as cattle (cows and bulls), sheep (lambs), pigs (piglet), goats and horses are meant. The method can be used to improve feed intake in any phase of the growth of a husbandry animal or may be used to increase feed intake by adding the feed composition during a determined and limited period during the life of the animals. The latter situation may occur when animals have poorer appetite because of sickness, after surgery or medical treatment, or in other stressful situations. In that case the method according to the description may suitably be used for pets and even humans as well.

Thus with the term “feed ingredients which have been adjusted to the specific needs of the animal concerned”, a combination of feed ingredients is meant that benefits both the nutritional as health requirements of the animal concerned in the specific situation (growth phase and/or condition) of the animal at that point in time.

Results are particularly beneficial when the method is applied early during the post-weaning period, in particular in the first week post-weaning.

We have found that piglets and pigs benefit from a smooth and problemless weaning period when using the method according to the description, which was shown by an improved feed conversion and in general healthier piglets.

However, as mentioned above the method may also suitably be used for other domesticated animals such as pets and for humans, provided that the ingredients have been adjusted for consumption of the animal or human concerned.

The present description is also directed to a method to improve feed intake of husbandry animals such as a piglet or a pig, a calf or cow or bull, a sheep or a lamb, a horse or a filly by offering a composition comprising benzocaine and feed ingredients according to the description.

Benzocaine is a known local anesthetic. It is sparingly soluble in water; it is more soluble in dilute acids and very soluble in ethanol, chloroform, and ethyl ether. The melting point of benzocaine is 88-90° C.

It is surprising that benzocaine is particularly effective in accomplishing this effect, because another local anesthetic was reported to reduce food intake when administered into the stomach (Pharmacology, Biochemistry and Behaviour, Vol 3, p 69-74, 1975). It seems, that other anesthetics may improve in feed intake action with coatings in order to mask the anesthetic from working in the mouth or interfere with swallowing or cause any other aversive taste. The lack of such coating may explain the earlier result describing food intake inhibiting effects of local anesthetics when administered in admixture with food (Op cit). Surprisingly, benzocaine apparently does not have such aversive properties.

Also our comparative experiments show that other local anestetics do not provide the positive result that the use of benzocaine provides.

The composition used in the method according to the description advantageously comprises benzocaine being present in a solid form, more advantageously in a solid particulate form. It was found that the benzocaine may suitably be added to the regular feed of the domesticated animals, provided it is adjusted to the specific needs of the animal concerned. Since benzocaine is solid at room temperature and usually exist in crystalline particulate form, it may easily be added to the regular animal feed in solid form. This facilitates setting the dosage for the farmer or pet-owner and also facilitates storage.

The benzocaine may be added to the feed ingredients without need for coating of the particles.

It goes without saying that the benzocaine may also be added to the feed ingredients in solution or in emulsified form, when circumstances desire.

The feed ingredients in the composition may comprise milk products, fibers and high quality protein source. Suitable milk products are wey, wey powder, lactose etcetera. High quality protein sources come from soya protein concentrate, potato protein, so as to simultaneously introduce small amounts of less digestible protein sources. These ingredients are also suitable for compositions for cattle, sheep and horses, but may be adjusted to the specific needs of the animal concerned and the specific situation of the animal, such as during weaning, post-surgery, post or during medical treatment, post or during sickness or other stressful situations.

When using the compositions for other domesticated animals or humans the ingredients may suitable by adjusted to the type of animal concerned. For instance for carnivorous pets, usually animal fats and proteins are present in addition to the benzocaine. For human application, any normal food ingredient can be used, but preferred are the ones that are easy to swallow and have high nutritious value. For instance the use of benzocaine in high-energy protein shakes of yoghurt is preferred for humans, because it is thought that the benzocaine also does not detrimentally affect the taste or texture of the food. The use of solid particulate benzocaine in liquid, emulsified, or suspended food ingredients, has the advantage of easy dose setting.

In the weaner phase, fiber becomes an important part of the diet. Fiber sources that are not or only poorly digestible (e.g. lignocellulose) can help to stabilize the gut, support gut development and contribute to a fast gut passage, not leaving pathogens much time to thrive.

The feed ingredients may further comprise feed additives such as enzymes, acidifiers or organic acids, probiotics, phytogenic feed additives or botanicals, and/or toxin binders and combinations thereof.

As mentioned-above, weaning feed is commercially available and comprises ingredients that are considered to improve the transition of mother's milk to solid feed.

In order to further stabilize the gut, weaning feeds usually make use of one or more the following feed additives:
Enzymes. Non-starch-polysaccharides (NSP) degrading enzymes reduce viscosity and increase the availability of nutrients in the digesta. Phytase degrades the phytic acid complexes and increases the availability of phosphorus and also other nutrients.
Acidifiers or organic acids. These help to increase the pH-value in the stomach. This increases protein degradation and also reduces the bacterial load in the digesta.
Probiotics. Beneficial bacteria stabilize the gut microbiota, by increasing the proportion of “good” bacteria to exclude pathogenic bacteria.
Phytogenic feed additives or botanicals. Consisting of plant-based substances e.g. essential oils, these have a wide range of different functions, from increasing feed intake due to better taste, to increasing protein digestibility and fighting pathogens.
Toxin binders. Mycotoxins and endotoxins can have detrimental effects on health and development of piglets. Avoiding these stress factors adds a lot to keeping piglets healthy during this vulnerable phase

The method according to the description is especially suitable for weaning, because as explained above it was found to diminish the weaning dip. The weaning feed can also be used for other infant animals. It was further found that the feed composition according to the description is also suitable for post-weaning feed.

A suitable dosage is selected to obtain effective feed intake increase depending on age and weight of the pig or piglets.

The amount of benzocaine present in the feed may be calculated to provide a dosage of 0.2-20 mg benzocaine per day per kg animal. For pigs or piglets this amounts to about 10-1000 mg benzocaine per day per piglet or pig.

The invention is also directed to a method to improve feed intake in a husbandry animal such as a piglet or a pig, a calf or cow or bull, a sheep or a lamb, a horse or a filly or foal, a goat or kid goat by offering feed in the form of a composition comprising benzocaine and feed ingredients according to the description.

The invention is further directed to a method to improve feed intake in a pet such as a dog or cat by offering a composition comprising benzocaine and feed ingredients according to the description.

As indicated above, this method has the advantage that the benzocaine can simply be added to the feed. This provides an elegant administration without further adding stress to the husbandry animal or pet.

The same advantage is provided when improving the food intake in a human by offering food in the form of a composition comprising benzocaine and food ingredients according to the description. Suitable food for humans may be shakes or yoghurts, because the benzocaine can be added without detrimentally affecting the taste and or texture of the food.

The method further pertains to offering composition comprising benzocaine and feed ingredients to the piglets during the weaning period. As explained above, it was found that this method decreases the weaning dip for pigs.

We do not have a complete and certain explanation for the mechanism of action of benzocaine in the present invention. It may be that satiety signals originating from the surface of the stomach and/or duodenum are blunted by benzocaine and that this incites the animals to postpone termination of a meal. Another possibility is that benzocaine reduces an aversive or repulsive signal arising from some constituents in food, and which constituents or signals would normally inhibit further intake or even initiate repulsion or sensations of aversion influencing the future perception of same or similar food. It is thought however that the modus of action is not based on alleviating pain before, during or after feed intake, because the test animals were not in pain.

Some observations of effects of local anesthetics on stomach in the context of food intake were described in the prior art, without the possibility to derive from those observations the effect disclosed in the present description. Uneyama et al. (Am. J. Physiol.—Gastrointestin. & Liver Physiol., Vol 291, pp 1163-1170, 2006) described chemosensing of glutamate in the stomach wall, signaled to the central nervous system by the gastric branch of the vagus nerve. Uneyama reported that this chemosensing signal can be blocked by lidocaine. However, it is known that glutamate in food induces overeating so Uneyama's result could suggest, if anything on food intake, a reduction of food intake by blunting the signal arising from chemosensing glutamate. Chee et al (Chemical Senses, Vol 30, pp 393-400, 2005) reported on observations on the effect of local anesthetic on the pharynx on eating and swallowing. It was observed that swallowing speed was reduced, swallowing interval prolonged and swallowing capacity unchanged, leading to the conclusion of the authors of this disclosure that chemosensory input influenced swallowing function.

The following examples are illustrative, but not limiting of the methods and compositions of the present disclosure.

### EXAMPLES

### Example 1 Weaning of Mice with Feed Comprising Various Local Anestetics

### Experimental Set-Up

Litters of mice are weaned on day 18 after birth. The siblings are divided over 2 groups (placebo and active) with group sizes of 7-10 siblings.

The pups are offered daily ad libitum food in which a local anesthetic is mixed. Body weight is measured daily.

### Results

In TABLES I-IV the body weight is depicted as percentage increase on day 14 in comparison to baseline, as a result of dosing lidocaine (comparative), prilocaine (comparative), bupivacaine (comparative) and benzocaine (according to the invention) to the mice. The baseline is the weight of the mouse at the beginning of the experiment. Placebo and experimental scores are statistically compared by means of student-t tests (* p<0,05; ** p<0,01; *** p<0,001). Doses are indicated on the x-axis and are mg per day for the group of siblings.

### Conclusion

Lidocaine, prilocaine and bupivacaine are either inactive (low dose) or inhibit the growth of the mice (in comparison to placebo). Benzocaine increases the growth at low doses (25, 50 and 100 mg) while it inhibits the growth at 200 mg.

### Example 2 Weaning of Piglets with Feed Comprising Various Amounts of Benzocaine

### Experimental Set Up

Six groups of 21 piglets were treated.

They were fed with controlled amounts of feed (see table V).

Before administration, the food pellets were drenched in water for 30 min and benzocaine (0 g (3×), 5 g, 12,75 g or 17 g, corresponding to a dose of 0, 30, 75 and 100 mg per day per pig, respectively) was added and mixed. The group of piglets receiving feed with 0% benzocaine is referred to as the placebo group.

Amounts of feed consumed were recorded. Piglets were weighed on day 1 (baseline), 4, and 8.

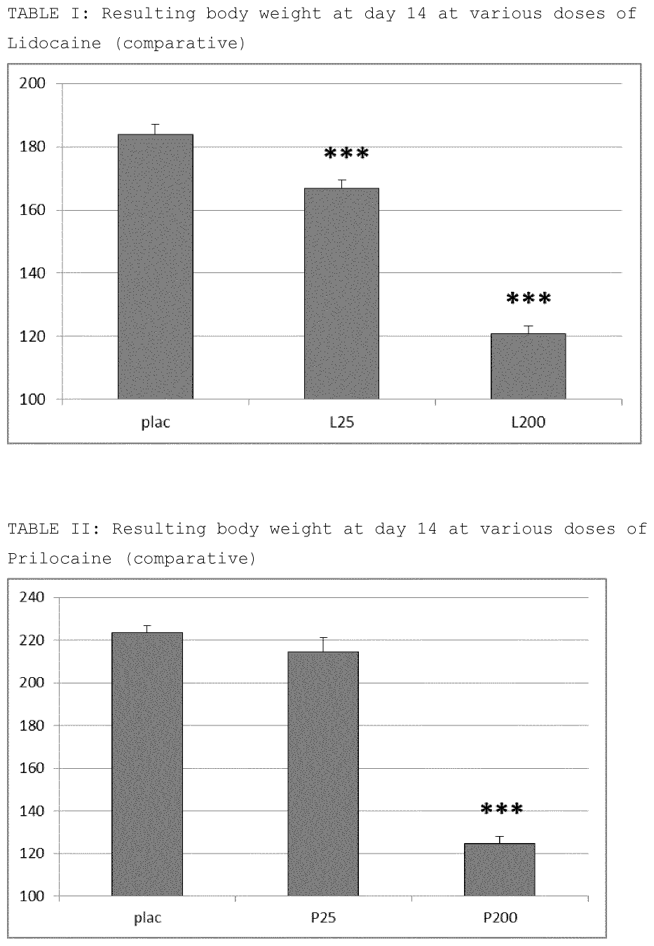

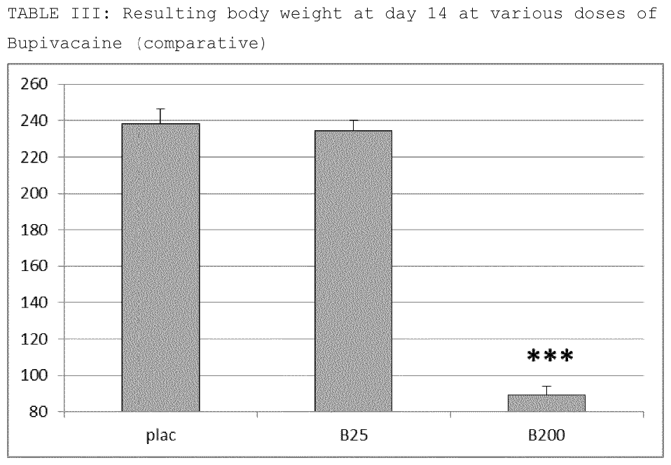

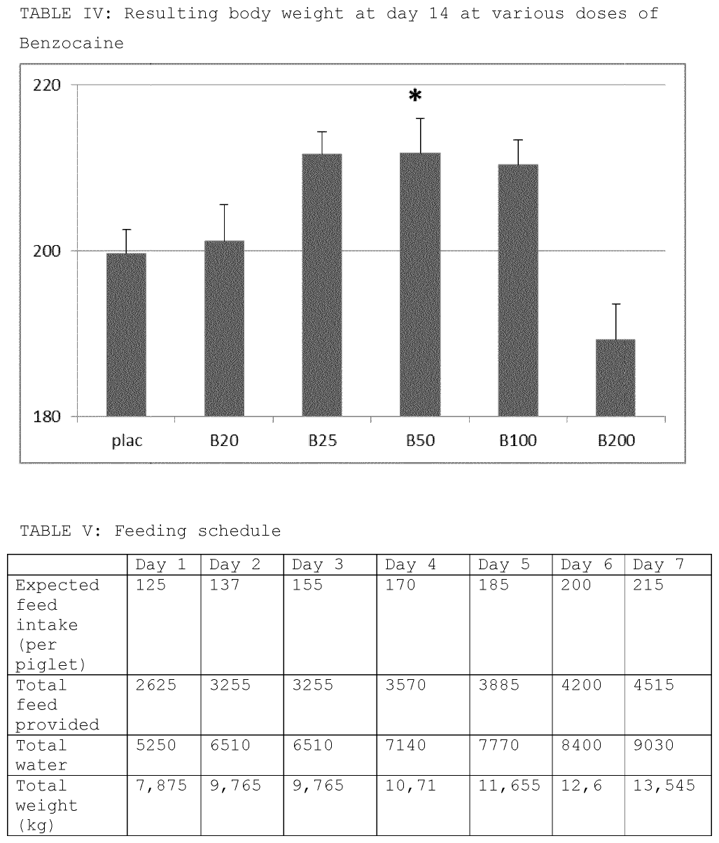

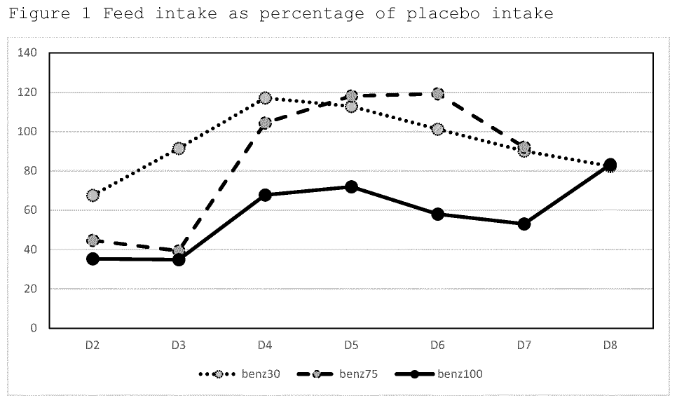

The feed intake as a percentage of the placebo intake is depicted in FIG. 1

FIG. 1 shows that in the first 3 days the pigs have an initial reduction of the feed intake, probably caused by the new taste of the feed or sedation of the gum, but after day 4 the pigs dosed with 30 mg and 75 mg of benzocaine per day show an increase feed intake, probably caused by a decreased satiety.

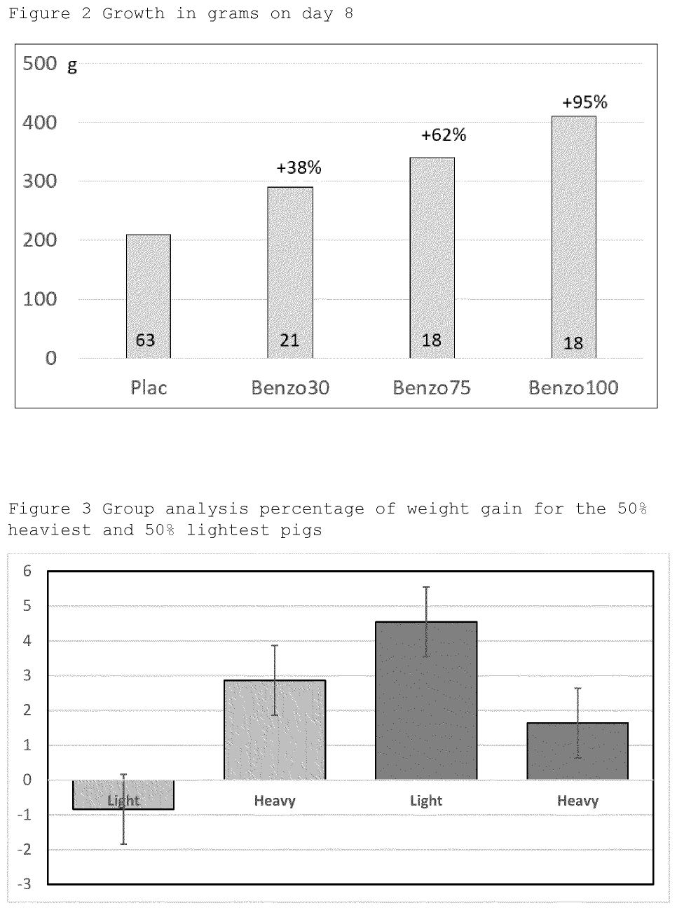

FIG. 2 gives the increase in weight for the pigs on day 8. The percentage at the top of each column gives the increase in weight as a percentage of weight increase of the pigs of the placebo group. The number in the columns show the number of pigs. 6 piglets died during the experiment.

In FIG. 3 the results of a group analysis of the percentage growth on day 7 of the lightest and heaviest 50% of the animals is given for the animals of the placebo group and the group provided benzocaine-containing feed.

These results show that the increase of growth in the benzocaine-fed group primarily takes place with the light weight animals. This means that a more homogenous group of pigs results.

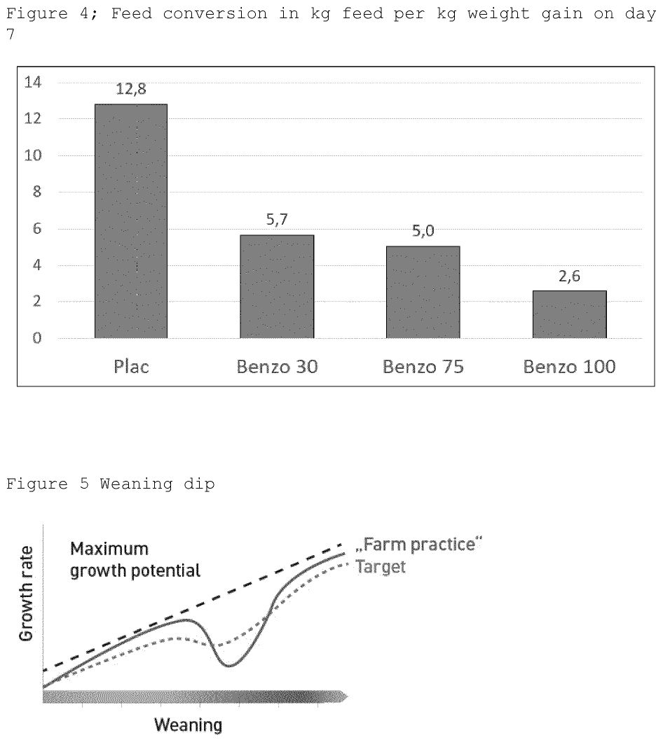

In FIG. 4 the feed conversion of the pigs is given as a percentage of weight gain per the feed intake. In the placebo group, i.e. fed with feed not containing benzocaine, about 12,8 kg feed was needed for 1 kg of weight gain (meat gain). This conversion improves with the increase of benzocaine dosing in the feed to 2,6 kg of feed per 1 kg of meat.

The optimal dose was found to be 75 mg per day. It was further found that the general condition of the benzocaine treated pigs was better than that of the placebo group. There was less fouling and less diarrhea.

1. A method to improve feed intake in a domesticated animal comprising a step of offering feed to the domesticated animal as a feed composition comprising benzocaine and feed ingredients that have been adjusted to specific needs of the domesticated animal.

2. The method according to claim 1, wherein the feed ingredients are adjusted to the specific needs of piglets or pigs.

3. The method according to claim 1, wherein the benzocaine is present in the feed composition in a solid, particulate form.

4. The method according to claim 1, wherein the feed ingredients comprises milk products, fibers and a high quality protein source.

5. The method according to claim 1, wherein the feed composition comprises one or more feed additives selected from the group consisting of enzymes, acidifiers or organic acids, probiotics, phytogenic feed additives or botanicals, toxin binders and combinations thereof.

6. The method according to claim 1, wherein the feed composition is weaning feed and is offered to infant animals during a weaning period.

7. The method according to claim 1, wherein the amount of the benzocaine present in the feed composition is calculated to provide a dosage of 0.2-20 mg benzocaine per day per kg of the domesticated animal.

8. The method according to claim 7, wherein the feed composition is provided to a pig or piglet and the amount of the benzocaine present in the feed composition is calculated to provide a dosage of 10-1000 mg benzocaine per day per piglet or pig.

9. A feed composition suitable for use in the method according to claim 1, comprising benzocaine and feed ingredients for domesticated animals or humans adjusted to the specific needs of the domesticated animals or the humans.

10. The feed composition according to claim 9 wherein the benzocaine is present in the feed composition in a solid, particulate form.

11. The feed composition according to claim 9, wherein the feed ingredients comprise milk products, fibers and a high quality protein source.

12. The method according to claim 2, wherein the benzocaine is present in the feed composition in a solid, particulate form.

13. The method according to claim 3, wherein the feed ingredients comprise milk products, fibers and a high quality protein source.

14. The method according to claim 13, wherein the feed composition comprises one or more feed additives selected from the group consisting of enzymes, acidifiers or organic acids, probiotics, phytogenic feed additives or botanicals, toxin binders and combinations thereof.

15. The method according to claim 14, wherein the amount of the benzocaine present in the feed composition is calculated to provide a dosage of 0.2-20 mg benzocaine per day per kg of the domesticated animal.

16. The method according to claim 15, wherein the feed ingredients are adjusted to the specific needs of piglets or pigs.

17. The method according to claim 15, wherein the feed composition is weaning feed and is offered to infant animals during a weaning period.

18. The method according to claim 6, wherein the feed composition is offered to piglets during a weaning period.

19. The method according to claim 17, wherein the feed composition is offered to piglets during a weaning period.

20. The feed composition according to claim 10, wherein the feed ingredients comprise milk products, fibers and a high quality protein source.
